(19) United States
(12) Patent Application Publication
(10) Pub. No.: US 2010/0272693 A1
(43) Pub. Date: Oct. 28, 2010

(54) BONE SCAFFOLDS, INJECTABLE BONE REPAIR MATERIALS AND METHODS FOR BONE REPAIR

(76) Inventors: EunAh Lee, Gyeonggi-do (KR); Eun Jeon, Seoul (KR); Pamela Robey, Bethesda, MD (US); Kyung Byung Yoon, Seoul (KR)

Correspondence Address: KLARQUIST SPARKMAN, LLP (OTT-NIH), 121 S.W. SALMON STREET, SUITE #1600, PORTLAND, OR, 97204-2988, US

(21) Appl. No.: 12/745,231

(22) PCT Filed: Oct. 22, 2008
(86) PCT No.: PCT/US08/12064

§ 371 Date: May 27, 2010

Related U.S. Application Data
(60) Provisional application No. 61/004,940, filed on Nov. 30, 2007.

(51) Int. Cl. A61L 27/54; A61K 47/48; A61P 19/00; A61K 33/42

(52) U.S. Cl. 424/93.7; 514/55; 424/602

(57) ABSTRACT

A method of bone repair that includes applying to a subject a composite that comprises at least one calcium phosphate, at least one polyamine polymer material; and a linking structure covalently crosslinking the calcium phosphate to the polyamine polymer material, wherein the linking structure is covalently bonded to the calcium phosphate via a —O—Si— moiety or an isocyanato moiety and the linking structure is covalently bonded to the polyamine polymer material via amine groups of the polyamine polymer. Also disclosed is a combination of ingredients comprising at least one polyamine polymer; and calcium phosphate particles that are substantially coated with at least one functional group that is covalently reactive with amine groups on the polyamine polymer; wherein the ingredients are adapted for injection into a subject for forming a bone replacement material in vivo.

### CROSS REFERENCE TO RELATED APPLICATION

This application claims the benefit of U.S. Provisional Application No. 61/004,940, filed Nov. 30, 2007, which is incorporated herein by reference in its entirety.

### FIELD

The disclosures herein relate to bone replacement materials and methods of using such materials.

### BACKGROUND

Bone formation or replacement is often a desired therapy for bone loss or defects due to fractures or bone degenerative diseases. A biomaterial for bone formation or replacement should (a) have sufficient mechanical load-bearing and impact strength to maintain structural integrity and (b) provide a suitable environment to induce new bone formation. A potentially ideal bone-replacement scaffold material would include an organic polymer for mechanical strength and ease-of-use and inorganic particles that participate in the bone mineralization pathway. However, it is hard to maintain the bone scaffold material as a homogeneous mixture because the organic polymer and the inorganic particles cannot be easily homogeneously mixed together.

### SUMMARY

Disclosed herein are several methods of bone repair.

In one embodiment, a method of bone repair is disclosed that includes applying to a subject a composite that comprises at least one calcium phosphate, at least one polyamine polymer material; and a linking structure covalently crosslinking the calcium phosphate to the polyamine polymer material, wherein the linking structure is covalently bonded to the calcium phosphate via a —O—Si— moiety or an isocyanato moiety and the linking structure is covalently bonded to the polyamine polymer material via amine groups of the polyamine polymer.

According to another embodiment, a method of bone repair includes introducing into a subject a composite that comprises at least one bone replacement material having surface-exposed hydroxyl groups; at least one polyamine polymer material; and a linking structure covalently crosslinking the calcium phosphate to the polyamine polymer material, wherein the linking structure is covalently bonded to the calcium phosphate via a —O—Si— moiety or an isocyanato moiety and the linking structure is covalently bonded to the polyamine polymer material via amine groups of the polyamine polymer.

A further disclosed bone repair method includes injecting into a subject at least one polyamine polymer and calcium phosphate particles that are substantially coated with at least one functional group that is covalently reactive with amine groups on the polyamine polymer.

Also disclosed herein is a composite, which may be used as a bone implant or repair material, comprising at least one calcium phosphate; at least one polyamine polymer material; and a linking structure covalently crosslinking the calcium phosphate to the polyamine polymer material, wherein the linking structure is covalently bonded to the calcium phosphate via a —O—Si— moiety or an isocyanato moiety and the linking structure is covalently bonded to the polyamine polymer material via amine groups of the polyamine polymer.

Also disclosed herein is a combination of ingredients comprising at least one polyamine polymer and calcium phosphate particles that are substantially coated with at least one functional group that is covalently reactive with amine groups on the polyamine polymer; wherein the ingredients are adapted for injection into a subject for forming a bone replacement material in vivo.

The foregoing and other objects, features, and advantages will become more apparent from the following detailed description, which proceeds with reference to the accompanying figures.

### BRIEF DESCRIPTION OF THE DRAWINGS

FIG. 1 depicts spectrums obtained from the absolute ethanol mixed with HA-APS-TPDA crystal powders before ninhydrin reaction (dotted line), after ninhydrin reaction (solid line), and the subtraction of the spectrum before-ninhydrin reaction from the spectrum after-ninhydrin reaction (solid line shown in smaller inset spectrum). A ninhydrin reaction confirms attachment of linker compound on the surface of HA. The HA powders refluxed with amino-propyl-tri-ethoxy-silane (APS) was subjected to a ninhydrin reaction. Presence of amine groups on surface of HA crystals was confirmed by Ruhemann's purple at wavelength of 405 and 570 nm.

FIG. 2A depicts a scheme for formulation of a bone sponge from HA-GPMS and gelatin.

FIG. 2B depicts a scheme for formulation of a bone sponge from HA-APS-TPDA and chitosan.

FIGS. 3A, 3B and 3C are scanning electron microscope (SEM) images of bone sponges made from HA-GPMS and gelatin (30×, 80×, and 30×, respectively).

FIGS. 3D, 3E and 3F are scanning electron microscope (SEM) images of bone sponges made from HA-APS-TPDA and chitosan (1,000×, 250×, and 150×, respectively).

FIG. 4 are photographs of the results of integrity testing of bone sponges in a submerged state. Bone sponges were made from untreated bare HA, and HA-GPMS-gelatin. Bone sponge samples were made with 9 mm punch and put in 6-well plate. Bone sponges were soaked with DW and tested for their structural integrity according to the time-course as indicated.

FIG. 5A is a graph depicting the results of a MTT assay that measures cell seeding efficiency. The MIT assay is described in more detail below, but in general various numbers of cells were seeded into HA-chitosan bone sponges and in 24-well plates, which were cultured for 24 hours. The cultures were treated with MIT dye for last 4 hours of culture. The cell-seeded sponges and 24 wells were washed with PBS and the bone sponges were crushed for extraction of MTT dye.

FIG. 5B is a photograph showing HA-chitosan bone sponges seeded with cells. Cell-seeded bone sponge was treated with MTT dye for 4 hours.

FIG. 6 shows SEM images of the in vitro cultured hBMSC-seeded sponges

FIG. 7 shows tissue sections obtained from in vivo transplantation of bone sponge with hBMSCs. The bone sponges made from HA-APS-TPDA and chitosan were seeded with hBMSCs and transplanted subcutaneously into immunocompromised mice for 10 weeks (A, B, C) and 15 weeks (D, E, F) as described in more detail below. Sections were H&E stained (A, D) and checked for autofluoresence (B, E) and polarized microscopy (C, F).

FIG. 8 shows in vivo transplantation of bone sponge without cells. The bone sponges made from HA-APS-TPDA and chitosan were soaked with fresh media and transplanted subcutaneously into immunocompromised mice for 15 weeks as described in more detail below. Sections were H&E stained (A) and checked for autofluorescence (B).

FIG. 9 is a schematic drawing of a reaction scheme to generate two inventive composites (HA-GPMS and HA-APS-TPDA).

### DETAILED DESCRIPTION

For ease of understanding, the following abbreviations and terms used herein are described below in more detail:

HA—hydroxyapatite

hBMSC—human bone marrow stromal cells

TPDA—terephthaldicarboxaldehyde

“Alkyl” refers to a branched or unbranched saturated hydrocarbon group.

“Alkoxy” refers to a radical of the formula —OR, wherein R is an alkyl.

“Amorphous,” when used in the context of mineral compositions, generally refers to a relatively unstructured, non-crystalline form of a mineral that is capable of acting as a seed and support for the growth of crystals thereon.

An “animal” is a living multicellular vertebrate organism, a category that includes, for example, mammals and birds. A “mammal” includes both human and non-human mammals. “Subject” includes both human and animal subjects.

“Biocompatible” refers to a material that, upon administration or implantation in vivo, does not induce undesirable long term effects.

“Biodegradable” refers to a material that degrades under physiological conditions. Biodegradable materials are not necessarily hydrolytically degradable and may require enzymatic action to fully degrade.

“Bioresorbable,” generally refers to a biocompatible material, composition or object that has the ability to be gradually integrated into a host. When used in the context of the subject prosthetic bone implants, the term generally refers to the ability of at least a portion of the prosthetic bone implant to gradually be replaced by natural bone, such replacement typically occurring naturally by the physiological process of bone remodeling. Thus, in the context of the presently described embodiments, the term “bioresorbable” is meant to include any material or process that is receptive to or typically associated with bone remodeling, including but not limited to osteoblast and osteoclast activity, deposition and/or mineralization of new bone matrix, vascular and cellular infiltration and tissue ingrowth.

“Crystalline” generally describes a mineral composition having relatively a well-defined crystal structure, with a unique arrangement of atoms within the component crystals. Pure hydroxyapatite typically crystallizes in the hexagonal crystal system, although alternate crystal structures may be realized by altering the composition of the mineral.

“Osteoconductive”, as used herein, refers to the ability of a substance or material to provide surfaces which are receptive to the growth of new bone.

“Osteoinductive,” when used in the context of a bioactive composition, generally refers to a composition that induces and/or supports the formation, development and growth of new bone, and/or the remodeling of existing bone. An osteoinductive composition typically includes one or more osteogenic agents. An “osteogenic agent,” as used herein, is an agent that can elicit, facilitate and/or maintain the formation and growth of bone tissue. Many osteogenic agents function, at least in part, by stimulating or otherwise regulating the activity of osteoblast and/or osteoclasts. Exemplary osteogenic agents include certain polypeptide growth factors, such as, osteogenin, Insulin-like Growth Factor (IGF)-1, TGFβ1, TGFβ2, TGFβ3, TGFβ4, TGFβ5, osteoinductive factor (OIF), basic Fibroblast Growth Factor (bFGF), acidic Fibroblast Growth Factor (aFGF), Platelet-Derived Growth Factor (PDGF), vascular endothelial growth factor (VEGF), Growth Hormone (GH), osteogenic protein-1 (OP-1) and any one of the many known bone morphogenic proteins (BMPs), including but not limited to BMP-1, BMP-2, BMP-2A, BMP-2B, BMP-3, BMP-3b, BMP-4, BMP-5, BMP-6, BMP-7, BMP-8, BMP-8b, BMP-9, BMP-10, BMP-11, BMP-12, BMP-13, BMP-14, BMP-15, and marrow stromal cells (e.g., human marrow stromal fibroblasts). An osteoinductive composition may include one or more agents that support the formation, development and growth of new bone, and/or the remodeling thereof. Typical examples of compounds that function in such a supportive manner include, though are not limited to, extracellular matrix-associated bone proteins (e.g., alkaline phosphatase, osteocalcin, bone sialoprotein (BSP) and osteocalcin in secreted phosphoprotein (SPP)-1, type I collagen, fibronectin, osteonectin, thrombospondin, matrix-gla-protein, SPARC, alkaline phosphatase and osteopontin).

The above term descriptions are provided solely to aid the reader, and should not be construed to have a scope less than that understood by a person of ordinary skill in the art or as limiting the scope of the appended claims.

The singular terms “a,” “an,” and “the” include plural referents unless context clearly indicates otherwise. Similarly, the word “or” is intended to include “and” unless the context clearly indicates otherwise. The word “comprises” indicates “includes.” Unless otherwise indicated, description of components in chemical nomenclature refers to the components at the time of addition to any combination specified in the description, but does not necessarily preclude chemical interactions among the components of a mixture once mixed.

A biocompatible bone replacement material that includes a strong covalent attachment between a bone substitute material (e.g., calcium phosphate) and a polyamine polymer matrix is disclosed herein. The covalent attachment enables forming and maintaining the bone substitute material and the polyamine polymer in a substantially homogeneous mixture. A substantially homogeneous mixture is evident from uniform composition, appearance, and properties throughout the mixture. For example, there is no, or only inconsequential, precipitation of the bone substitute material from the mixture. In certain embodiments, the bone replacement material is osteoinductive. The bone replacement material also may be at least partially bioresorbable.

In one embodiment, the covalent attachment is formed via a first bifunctional linker compound covalently bonded to functional groups (preferably hydroxyl) exposed on the surface of the calcium phosphate material. The first linker compound also includes a second functional group that is reactive with a second bifunctional linker compound such that the calcium phosphate material is substantially coated with the second linker compound. The second bifunctional linker includes a first moiety that is reactive with the first linker compound and a second moiety that is reactive with the polyamine polymer. The functional group-coated calcium phosphate (also referred to herein as “surface-modified” calcium phosphate) is reacted with the polyamine polymer resulting in a bone replacement or filler material.

In a second embodiment, the covalent attachment is formed via a single bifunctional linker compound (instead of a two linker compound system described above). The single bifunctional linker compound includes a first reactive group that can covalently bond to functional groups exposed on the surface of the calcium phosphate material. The single bifunctional linker compound also includes a second reactive group that can covalently bond to the polyamine polymer.

The calcium phosphate materials may be naturally occurring or, preferably, they are synthetic. The calcium phosphate may have an amorphous or, preferably, a crystalline structure. The calcium phosphate as mixed with the polyamine polymer may be in the form of a powder that includes individual calcium phosphate crystals or crystalline particles, or granules of various sizes, or blocks, or sponges. Illustrative calcium phosphates have the general chemical formula Ca₅(PO₄)₃X, where X is OH (hydroxyapatite), F (fluorapatite), or Cl (chlorapatite). Such materials are also known as “apatites.” The term “hydroxyapatite” or “HA” as used herein, generally refers to a form of apatite with the formula Ca₅(PO₄)₃(OH), but is more typically represented as Ca₁₀(PO₄)₆(OH)₂ to denote that the crystal unit cell comprises two molecules. Hydroxylapatite is the hydroxylated member of the complex apatite group. The hardness of hydroxyapatite may be altered by replacing the OH ion with other anions (e.g., fluoride, chloride or carbonate). Additionally, HA has a relatively high affinity for peptides, making it an ideal carrier for the delivery and sustained release of polypeptides over long periods of time in situ.

Other bone substitute materials may also be used, provided they have, or are treated to have, reactive hydroxyl functional groups present on or at the surface of the material. Such materials include aragonite, dahlite, calcite, amorphous calcium carbonate, vaterite, weddellite, whewellite, struvite, urate, ferrihydrite, francolite, monohydrocalcite, magnetite, goethite, dentin, calcium carbonate, calcium sulfate, calcium phosphosilicate, sodium phosphate, calcium aluminate, α-tricalcium phosphate, dicalcium phosphate, β-tricalciumphosphate, tetracalcium phosphate, amorphous calcium phosphate, octacalcium phosphate, BIOGLASS™, magnesium-substituted tricalcium phosphate, carbonate hydroxyapatite, substituted forms of hydroxyapatite (e.g., hydroxyapatite may be substituted with other ions such as fluoride, chloride, magnesium, sodium, potassium, etc.), and combinations and derivatives thereof. In certain embodiments, the particles themselves are composites that include one or more of an inorganic material, a bone substitute material, and a bone-derived material; and one or more of bovine serum albumin, collagen, an extracellular matrix component, a synthetic polymer, and a natural polymer. A mixture of different bone substitute materials may be used.

The polyamine polymer for the composite may be any biocompatible polymer that includes reactive amine functional groups, particularly primary amine groups on every residue or every other residue. The polyamine polymer's molecular weight ranges from 50,000 to 1,000,000 and more desirably from 650,000 to 1,000,000. For example, the polyamine polymer may be a polysaccharide having primary amine functionality. Illustrative amine-functional polysaccharides include glucosamine-containing polymers such as chitosan and partially deacetylated chitin. Other polyamine polymers include collagen (particularly microcrystalline collagen), gelatin, polyglutamate, any synthetic polypeptides that contain at least one amine group in each residue, chondroitin sulfate and proteoglycans. A mixture of different polyamine polymers may be used.

“Chitosan” is inclusive of chitosan per se, modified chitosans, crosslinked chitosan and chitosan salts. Chitosan is an aminopolysaccharide usually prepared by deacetylation of chitin (poly-beta(1,4)—N-acetyl-D-glucosamine). Chitin occurs widely in nature, for example, in the cell walls of fungi and the hard shell of insect and crustaceans. The waste from shrimp-, lobster, and crab seafood industries typically contains about 10 to about 15 percent chitin and is a readily available source of supply. In the natural state, chitin generally occurs only in small flakes or short fibrous material, such as from the carapace or tendons of crustaceans. There is generally no source that forms useful shaped articles without solution and re-precipitation or re-naturing.

When many of the acetyl groups of chitin are removed by treatment with strong alkalis, the product is chitosan, a high molecular weight linear polymer of 2-deoxy-2-amino glucose. Chitosan typically is not a single, definite chemical entity but varies in composition depending on the conditions of manufacture. It may be equally defined as chitin sufficiently deacetylated to form soluble amine salts.

The chitosan used herein is suitably in relatively pure form. Methods for the manufacture of pure chitosan are well known. Generally, chitin is milled into a powder and demineralized with an organic acid such as acetic acid. Proteins and lipids are then removed by treatment with a base, such as sodium hydroxide, followed by chitin deacetylation by treatment with concentrated base, such as 40 percent sodium hydroxide. The chitosan formed is washed with water until the desired pH is reached.

The properties of chitosan generally relate to its polyelectrolyte and polymeric carbohydrate character. Chitosan generally dissolves readily in dilute solutions of organic acids such as formic, acetic, tartaric, glycolic, lactic and citric acids, and also in dilute mineral acids, except, for example, sulfuric acid. In general, the amount of acid required to dissolve chitosan is approximately stoichiometric with the amino groups. Since the pKa for the amino groups present in chitosan is between 6.0 and 7.0, they can be protonated in very dilute acids or even close to neutral conditions, rendering a cationic nature to this biopolymer.

Certain chitosan materials for use herein have an average degree of deacetylation (D.A.) of more than 70%, preferably from 80% to about 100%, even more preferably from 90% to 100% and most preferably from 95% to about 100%. The degree of deacetylation refers to the percentage of the amine groups that are deacetylated. This characteristic is directly related to the hydrogen bonding existing in this biopolymer, affecting its structure, solubility and ultimately its reactivity. The degree of deacetylation can be determined by titration, dye adsorption, UV-VIS, IR, and NMR spectroscopy.

Suitable chitosan materials include both water-soluble and water insoluble chitosan. As used herein, a material will be considered to be water-soluble when it substantially dissolves in excess water to form a clear and stable solution, thereby, losing its initially particulate form and becoming essentially molecularly dispersed throughout the water solution. Particularly suitable chitosan materials for use herein are water soluble, i.e., at least 0.5 gram, preferably at least 1 gram and most preferably at least 2 grams of the chitosan materials are soluble in 100 grams of water at 25° C. and one atmosphere. By “solubility” of a given compound it is to be understood herein the amount of said compound solubilized in de-ionized water at 25° C. and one atmosphere in the absence of precipitate. As a general rule, the water-soluble chitosan materials will be free from a substantial degree of crosslinking, as crosslinking tends to render the chitosan materials water insoluble.

Chitosan (i.e., chitosan and chitosan salts, modified chitosans and cross-linked chitosans) may generally have a wide range of molecular weights. For example, useful chitosan typically has a molecular weight (weight average) ranging from about 150,000 to about 1,000,000, more particularly about 700,000 to about 1,000,000.

A variety of acids can be used for forming aminopolysaccharide salts like chitosan salts. Suitable acids for use are soluble in water or partially soluble in water, are sufficiently acidic to form the ammonium salt of the aminopolysaccharide and yet not sufficiently acidic to cause hydrolysis of the aminopolysaccharide, and are present in amount sufficient to protonate the reactive sites of the deacetylated aminopolysaccharide. Preferred acids can be represented by the formula: R—(COOH)ₙ wherein n has a value of 1 or 2 or 3 and R represents a mono- or divalent organic radical composed of carbon, hydrogen and optionally at least one of oxygen, nitrogen and sulfur or R is simply a hydroxyl group. Preferred acids are the mono- and dicarboxylic acids composed of carbon, hydrogen, oxygen and nitrogen (also called herein after amino acids). Such acids are highly desired herein as they are biologically acceptable for use against or in proximity to the human body. Illustrative acids, in addition to those previously mentioned include, among others, citric acid, formic acid, acetic acid, N-acetylglycine, acetylsalicylic acid, fumaric acid, glycolic acid, iminodiacetic acid, itaconic acid, lactic acid, maleic acid, malic acid, nicotinic acid, 2-pyrrolidone-5-carboylic acid, salicylic acid, succinamic acid, succinic acid, ascorbic acid, aspartic acid, glutamic acid, glutaric acid, malonic acid, pyruvic acid, sulfonyldiacetic acid, benzoic acid, epoxysuccinic acid, adipic acid, thiodiacetic acid and thioglycolic acid. Any aminopolysaccharide salt, especially chitosan salts formed from the reaction of the aminopolysaccharide with any of these acids are suitable for use herein.

Examples of chitosan salts formed with an inorganic acid include, but are not limited to, chitosan hydrochloride, chitosan hydrobromide, chitosan phosphate, chitosan sulphonate, chitosan chlorosulphonate, chitosan chloroacetate and mixtures thereof. Examples of chitosan salts formed with an organic acid include, but are not limited to, chitosan formate, chitosan acetate, chitosan lactate, chitosan glycolate, chitosan malonate, chitosan epoxysuccinate, chitosan benzoate, chitosan adipate, chitosan citrate, chitosan salicylate, chitosan propionate, chitosan nitrilotriacetate, chitosan itaconate, chitosan hydroxyacetate, chitosan butyrate, chitosan isobutyrate, chitosan acrylate, and mixtures thereof. It is also suitable to form a chitosan salt using a mixture of acids including, for example, both inorganic and organic acids.

Other aminopolysaccharide materials suitable for use herein include cross-linked aminopolysaccharides and modified aminopolysaccharides, especially cross-linked chitosans and modified chitosans.

Any type of gelatin that can provide the desired structural strength and reactive functional groups may be used. Such gelatins include, for example, alkali-treated gelatin (cattle bone or hide gelatin), acid-treated gelatin (pigskin or bone gelatin), and gelatin derivatives such as partially phthalated gelatin, and acetylated gelatin. Cross-linked gelatins which are cross-linked with a conventional cross-linking agent such as an aldehyde (e.g., formaldehyde or glutaldehyde) may also be used. Such compositions often include other components such as a medicament or a second polymer such as collagen or starch.

There are three general classes of collagen that are typically useful as bone implant materials. These include collagen-based implants comprised of soluble collagen, reconstituted collagen fibers, or natural insoluble collagen fibers. “Soluble collagen” refers to the solubility of individual tropocollagen molecules in acidic aqueous environments. Tropocollagen may be considered the monomeric unit of collagen fibers and its triple helix structure is well recognized. “Reconstituted collagen” is essentially collagen fiber segments that have been depolymerized into individual triple helical molecules, then exposed to solution and then re-assembled into fibril-like forms. Therefore, the degree of polymerization of reconstituted collagen is between that of soluble and native insoluble fibrous collagen. A disadvantage of reconstituted collagen is, in general, the mechanical strength and in vivo persistence are inferior to native (i.e. natural) insoluble fibrous collagen. “Natural insoluble collagen” refers to collagen that cannot be dissolved in an aqueous alkaline or in any inorganic salt solution without chemical modification, and includes for example hides, splits and other mammalian or reptilian coverings. For example, “natural insoluble collagen” can be derived from the corium, which is the intermediate layer of a animal hide (e.g. bovine, porcine, etc.) that is situated between the grain and the flesh sides.

The composite may also include (e.g., incorporated into the material matrix via chemical bonding or physical mixture) other components. For example, the composite may further include one or more of an initiator, accelerator, catalyst, solvent, wetting agent, lubricating agent, labeling agent, plasticizer, radiopacifier, porogen, bioactive agent, biostatic agent, cell, polynucleotide, protein (e.g., bone morphogenic protein, cytokine, growth factor, angiogenic factor), pharmaceutical agent (e.g., anti-inflammatory agent, analgesic, antibiotic, etc.), and pharmaceutically acceptable excipient. In certain embodiments, the composite includes a plasticizer that softens the composite making it more pliable. Exemplary plasticizer include glycerol and poly(ethylene glycol) (PEG) (e.g., PEG 8000, PEG 6000, PEG 4000). In certain embodiments, the polymer component of the composite includes PEG blended, grafted, or co-polymerized with the polymer. In certain embodiments, the composite includes a porogen that diffuses, dissolves, and/or degrades after implantation of the composite leaving a pore. The porogen may be a gas (e.g., carbon dioxide, nitrogen), liquid (e.g., water), or solid (e.g., crystalline salt). The porogen may be a water-soluble chemical compound such as a carbohydrate (e.g., poly(dextrose), dextran), salt, polymer (e.g., polyvinyl pyrrolidone), protein (e.g., gelatin), pharmaceutical agent (e.g., antibiotics), small molecule, etc.

In certain embodiments, the composite may include an osteogenic agent(s) such as mesenchymal stem cells (e.g, human bone marrow stromal cells) as described, for example, in U.S. Pat. No. 5,914,121, which is incorporated herein by reference. The bone marrow stromal cells can be included in the range of about 1 million cells/40 mg composites to about 5 million cells/40 mg composites.

The bifunctional linker compound is a material that mediates the linkages between the bone replacement material and the polyamine polymer. The compound should include at least one first functional group capable of forming a covalent bond with functional groups of the bond replacement material (particularly hydroxyl groups that are exposed on or at the surface of the bone replacement material). The compound should also include at least one second functional group capable of forming a covalent bond (i) with a second bifunctional compound or (ii) directly with functional groups of the polyamine polymer. In the two bifunctional compound embodiment, the second bifunctional compound includes a functional group that reacts directly with functional groups of the polyamine polymer.

The functional group capable of forming a covalent bond with the bone replacement material may be a silyl group, an alkoxysilyl group (e.g., —Si(OR₃)₃ wherein R is an alkyl group such as methyl or ethyl), or an isocyanato group (—N═C═O). Illustrative silyl groups include halosilyl (e.g., —SiX₃₋ₙAₙ (n=0-2) wherein X=halogen atom, A=alkyl, hydrogen, alkoxy or aryl groups) and siloxy (e.g., —OSiH₃). The functional group capable of forming a covalent bond with the polyamine polymer may, for example, be a group such as carbonyl (i.e., an aldehyde or ketone), carboxyl, or epoxy that can react with the amine groups of the polyamine polymer.

In illustrative embodiments, the bifunctional linker compound may be represented by the following formulae 1-5:

R₃Si-L-X (Formula 1)

R₃Si-L-Y (Formula 2)

Y-L-Y (Formula 3)

Y—Y (Formula 4)

R₃Si-L (Formula 5)

wherein, R represents a halogen atom, or C₁-C₄ alkoxy or alkyl group, provided at least one of the three R groups is a halogen atom or an alkoxy group; L represents substituted or unsubstituted C₁-C₁₇ alkyl, aralkyl or aryl group which may have at least one oxygen, nitrogen and sulfur atom; X represents a leaving group selected from the group consisting of halogen, isocyanate, tosyl and azide, most preferably, halogen; Y represents a reactive functional group of coordinate compounds capable of exchanging ligands selected from the group consisting of hydroxyl, thiol, amine, ammonium, sulfone and its salt, carboxyl acid and its salt, acid anhydride, epoxy, aldehyde, ester, acrylate, isocyanate (—NCO), sugar residue, double bond, triple bond, diene, diyne and alkylphosphine in which said reactive functional group may be present in the middle or at the terminal ends of the bifunctional linker compound. Examples of bifunctional linker compounds include aminopropyl-triethoxysilane, aminopropyl-trimethoxysilane, (3-cyanopropyl)-trichlorosilane (CNP-TCS), 1,4-diisocyanatobutane (DIC-4), 1,6-diisocyanatohexane (DIC-6), tolylene 2,4-diisocyanate-terminated poly(1,4-butanediol), isophorone diisocyanate-terminated poly(1,4-butanediol) terephthaldicarboxaldehyde (TPDA), and glycidoxypropyl trimethoxysilane (GPMS) (which can be used in the single linker compound approach).

The bifunctional linker compound combined to the bone replacement material may have at least one functional group in the skeleton of the linker compound to give the secondary chemical linkage to the polyamine polymer. For instance, where a linker compound combined to calcium phosphate contains formyl groups (—CHO), the calcium phosphate can chemically bind to chitosan since a chemical reaction between the amino groups of chitosan and the formyl groups may easily occur.

The functional group reaction methodology between the first functional group of the bifunctional linker compound and the functional group of the bone replacement material is well known. Similarly, the functional group reaction methodology between the second functional group of the bifunctional linker compound and (i) the first functional group of a second linker compound or (ii) the functional group of the polyamine polymer is well known. Advantageously, these reactions should proceed at room temperature and in water without generating any toxic or harmful by-products or heat.

In one embodiment, the first bifunctional linker compound is aminopropyl-trimethoxysilane (APS). The silyl groups of APS react with hydroxyl groups on the surface of the HA crystals. The amino groups of APS react with a second bifunctional linker compound—terephthaldicarboxaldehyde (TPDA). The resulting material is aldehyde-coated HA crystals. The aldehyde-coated HA then is reacted with the amino groups of a polyamine polymer (e.g., chitosan) to form a crosslinked biocomposite with the HA covalently bonded to the chitosan. The aldehyde-coated HA crystals are an effective crosslinker for forming a crosslinked, self-assembled, polymer network between the HA crystals and the chitosan matrix. The reaction between the aldehyde-coated HA crystals and the chitosan does not produce any harmful chemicals or heat and proceeds at room temperature and in water. This means that the biocomposite material can be utilized as an injectable, gel-forming material in which the aldehyde-amine reaction occurs in vivo. In addition, the reaction between the aldehyde groups and the amino groups is very fast and the yield is almost 100%. Amine-coated or epoxy-coated HA crystals can be synthesized in a similar fashion.

The amount of bone substitute material (e.g., calcium phosphate) in the composite should be appropriate to provide a suitable environment to induce new bone formation. For example, the amount of bone substitute material may range from about 50 weight % to about 98 weight %, particularly about 80 weight % to about 98 weight %, and more particularly about 90 weight % to about 95 weight %, based on the combined weight of the bone substitute material and the polyamine polymer. The amount of polyamine polymer in the composite should be appropriate to provide sufficient mechanical load-bearing and impact strength to maintain structural integrity. For example, the amount of polyamine polymer may range from about 1 weight % to about 50 weight %, particularly about 2 weight % to about 20 weight %, and more particularly about 5 weight % to about 10 weight %, based on the combined weight of the bone substitute material and the polyamine polymer.

The biocomposite material can be used, for example, as an injectable bone-filling material, an artificial bone sponge for ameliorating bone defects, or an artificial bone sponge for bone cell culture in bone and mineralization research.

The composite may be used as a bone implant to replace a portion of a human bone or bone system, cartilage, or teeth. For example, the bone implant may be used to repair a fracture or other bony defect in a subject's bone. Bone implants are often applied at a bone defect site, e.g., one resulting from injury, defect brought about during the course of surgery, infection, malignancy, inflammation, or developmental malformation. Bone implants can be used in a variety of orthopedic, neurosurgical, dental, and oral and maxillofacial surgical procedures such as the repair of simple and compound fractures and non-unions, external, and internal fixations, joint reconstructions such as arthrodesis, general arthroplasty, deficit filling, disectomy, laminectomy, anterior cerival and thoracic operations, spinal fusions, etc. The bone implant may have a shape that allows the implant to match the bone that the implant is used to replace. A bone implant may have a circular, oval, elongated disk, ring, square, rectangular, or irregular cross-sectional shape. In other embodiments, a bone implant may be U-shaped, C-shaped, an elongated ring with a gap, a disk with an orifice, or an elongated disk with an orifice.

The bone implant material could be molded, shaped, or injected into the site of implantation and then gelled under predetermined suitable conditions such as temperature ranges from room temperature to human body temperature and pH ranges from weak acid to weak base. The pH may range from about 5 to about 9, more particularly about 6 to about 7.4.

The particles and polyamine polymer may be mixed together to form a pre-gelled mixture. The pre-gelled mixture of the particles and the polymer can be made moldable or flowable such as by heating and/or the addition of a solvent. The pre-gelled mixture may range from a thick, flowable liquid to a moldable, dough-like substance. In other embodiments, the pre-gelled mixture is workable so that it can be molded into an implantation site.

In one preferred embodiment, the functionalized calcium phosphate particles and the polyamine polymer are injected into a subject. The particles and polyamine polymer may be mixed shortly before injection to form a pre-gelled mixture. The pre-gelled mixture then is injected into the subject. The functionalized particles may be suspended in any type of clinically-acceptable liquid carrier such as a saline solution (e.g., phosphate-buffered saline) prior to mixture with the polyamine polymer. The polyamine polymer may also be dissolved or suspended prior to mixing. Gellation of the mixture occurs about 5 to about 30 minutes after mixing of the particles and the polyamine polymer. The gellation time depends on the concentration of each component, pH and temperature.

Alternatively, the particles and the polyamine polymer may be injected separately with the mixing occurring in vivo. The particles and polyamine polymer may be dissolved or suspended in any type of clinically-acceptable liquid carrier. The injected mixture (either the pre-gel mixture or the separately administered particles and polymer) undergoes in vivo gel formation. The injected mixture can conform to the microstructure of the injection site. A further advantage is that bone seeding cells (and/or osteogenic agent(s)) can be mixed homogeneously into an injectable material.

In certain embodiments, the gelled composite has a micro-porous and/or macro-porous matrix structure composed of calcium phosphate particles crosslinked via a polyamine polymer network. The composite may have a biphasic pore distribution including both micropores and macropores. For example, the pore size may range from about 5 μm to about 1 mm, with the micropores ranging from about 5 μm to about 100 μm and the macropores ranging from about 200 μm to about 1 mm.

The composites may also serve as osteoconductive scaffolds for supporting new bone formation. The surface-modified bone substitute material (e.g., the functional group-coated HA crystals) functions as a cross-linker resulting in a homogeneous artificial bone scaffold. As explained below in the Examples section, when hBMSCs were seeded within this scaffold, there was good cell survival and distribution.

In another embodiment, the invention provides kits for the treatment of bone. The kit includes a composition including a plurality of particles including one or more of an inorganic material, a bone substitute material, and a bone-derived material; and a polymer with which the particles are combined, the composition being contained within a delivery system for delivering the composite by injection (e.g., a syringe). The kit may also include a high pressure injection device for implanting composite of higher viscosity. The injection device may operate by hydraulic or pneumatic means. The kit may also include the components of the composite packaged separately for mixing just prior to implantation. The composite is preferably sterilely packaged. In certain embodiments, the entire kit is sterilely packaged for use in a sterile environment such as an operating room. Various amounts of the composite may be packaged in a kit. For larger implantation sites, kits with greater amounts of composite are used. The amount of composite packaged in a kit may depend on the procedure being performed on the subject. In certain embodiments, multiple individually packaged amounts of composite are included in one kit. That way only the necessary number of packages need be opened for a procedure. The kit may also include a solvent or pharmaceutically acceptable excipient for combining with the composite. The kit may further include instructions for using the composite.

### EXAMPLES

### Materials

Commercial hydroxyapatite (HA) and glutaraldehyde (GA) was purchased from Sigma-Aldrich (St. Louis, Mo.). Flaked high molecular weight chitosan was a gift from Drs., Young Sook San and Young Ju Choi in Modern Tissue Technology, and molecular weight and deacetylation degree are 6˜7×10⁵ g/mol and 85%, respectively. Aminopropyl-trimethoxysilane (APS), glycidoxypropyl trimethoxysilane (GPMS), terephthaldicarboxaldehyde (TPDA) were purchased from Sigma-Aldrich (St. Louis, Mo.) and used as received.

### Preparation of Chitosan Solution

Chitosan solution was prepared by dissolving 1 g of chitosan flakes in 100 ml of 1% (v/v) acetic acid solution. The solution was stirred overnight and filtered prior to use.

### Preparation of HA-APS-TPDA

Preparation of hydroxyapatite crystals tethered with APS. Commercial hydroxyapatite was calcined and treated with APS (57.3 mM, 100 ml, 110° C., 1 h) under argon. The resulting hydroxyapatite crystals tethered with APS were then washed several times with fresh toluene, ethanol, and subsequently with methanol. The presence of surface-bound 3-aminopropylsilyl groups was confirmed by chromogenic reaction when surface-modified hydroxyapatite crystals were treated with ninhydrin in ethanol. The UV/vis spectrum of the purple colored hydroxyapatite showed an absorption at 570 nm indicating the presence of amine groups on hydroxyapatite crystals.

Preparation of hydroxyapatite crystals tethered with APS-TPDA. TPDA (3 g) was dissolved in toluene. The hydroxyapatite crystals tethered with APS were dispersed in toluene, and added to the vigorously stirred TPDA solution by dropping with a micropipette. The mixture was refluxed at 120° C. for 2 h under argon. After cooling to room temperature, the resulting hydroxyapatite crystals tethered with APS-TPDA were then washed several times with fresh toluene, ethanol, and subsequently with methanol. The HA-APS-TPDA powders were dried by vacuum evaporation. The presence of the TPDA on hydroxyapatite surface was confirmed by UV/vis absorption spectrum at 267 nm due to the presence of a phenyl moiety. Preparation of hydroxyapatite crystals tethered with GPMS. Commercial Hydroxyapatite was treated with GPMS (57.3 mM, 100 ml, 110° C., 1 h) under argon. The resulting hydroxyapatite crystals tethered with GPMS were then washed several times with fresh toluene, ethanol, and subsequently with methanol. The presence of the surface-bound glycidoxy groups was confirmed by testing the composite's ability to maintain structural integrity in water.

### Formation of Composite Material

Formation of HA-gelatin composite. 1 ml of 20% gelatin (equivalent to 200 mg of dry gelatin) solution was mixed with 1 ml of distilled water dispersed with 1 g HA-GPMS powder. The mixture was left standing for 30 min at room temperature for completion of the cross-linking reaction. After completion of the reaction, the wet composite was frozen at −20° C. and dried by freeze-drying.

Formation of HA-chitosan composite. 2.5 ml of 1% chitosan (equivalent to 25 mg of dry chitosan) solution was mixed with 2.5 ml of distilled water dispersed with 500 mg HA-APS-TPDA powder. The mixture was aliquoted in a volume of 500 μl to 48-well to make 10 composites and left standing for 30 min at room temperature for completion of the cross-linking reaction. After completion of the reaction, the wet composite was frozen at −20° C. and dried by freeze-drying.

### Scanning Electron Microscopy

Scanning electron microscope (SEM) images of HA-gelatin and HA-Chitosan composites were obtained with a FE-SEM (Hitachi S-4300) at an acceleration voltage of 14 kV.

The composites were washed with deionized water and methanol, then vacuum-dried. For the hBMSC-seeded sponges, the cell-seeded sponges were rinsed with PBS three times and fixed with 4% paraformaldehyde for 1 h at room temperature, and dehydrated in 70%, 80%, 90%, 95% and absolute ethanol for 30 min each, respectively. The samples were then dried and platinum-coated using a sputter coater for 90 sec to make platinum deposited with a thickness of about 15 nm on surface before SEM observation.

### Cell Culture and MTT Assay

Surgical specimens were obtained with fragments of normal unaffected bone with bone marrow from patients undergoing reconstructive surgery. Tissue procurement proceeded in accordance with institutional regulations governing the use of human subjects, including the use of informed consent. Multicolony strains of bone marrow stromal cells were derived from the bone marrow in a manner previously described (Kuznetsov S A et al., 1997 JBMR). Briefly, a single cell suspension of bone marrow cells was cultured in growth medium consisting of α-MEM, 2 mM L-glutamine, 100 U/mL penicillin, 100 μg/mL streptomycin sulfate, 10⁻⁸ mol/L dexamethasone, 10⁻⁴ mol/L L-ascorbic acid phosphate magnesium salt n-hydrate, and 20% fetal bovine serum of a preselected lot. The cells were then incubated at 37° C. in an atmosphere of 100% humidity and 5% CO₂. After 1 day, nonadherent cells were removed by means of extensive washing.

Cells were passaged at near confluence with trypsin-EDTA (Invitrogen; Carlsbad, Calif.). Upon reaching confluence at passages 2 through 5, cells were released by trypsin-EDTA and seeded to the HA-chitosan composites in 24-well at a density as indicated, and incubated for 1 day prior to MTT assay. MTT assay was performed as described (Mosmann, T., et al., 1983) As a control, hBMSCs were seeded to 24-wells without composites at the same density and incubated for 1 day before MTT assay.

In Vivo Transplantation of Bone Sponges with or without BMCSs

Osteoinductivity of HA-chitosan composite was analyzed by in vivo transplantation assay. HA-chitosan composites were seeded with polyclonal hBMSCs as described (Kuznetsov S A, et al., 1997, JBMR). Briefly, 2×10⁶ cells in 1 ml of culture medium were mixed with 50 mg of sterile HA-chitosan composite in a 1.8 ml cryotube. After 90-min incubation at 37° C., the particles with attached cells were collected by brief centrifugation. Eight to 15 week-old immunodeficient female beige mice (bg-nu/nu-xid, Harlan Sprague-Dawley, Indianapolis, Ind.) served as transplant recipients. All animals were cared for according to the policies and principles established by the Animal Welfare Act and the NIH Guide for the care and use of laboratory animals. Mice were anesthetized with isoflurane inhalation. Transplants were placed in subcutaneous pockets in the back of mice. Incisions were closed with stainless steel surgical staples. Four transplants per mice were generated, and each experiment was repeated at least three times. The transplants were recovered according to the time course as indicated and the results (6, 8, 10, 12, 15 weeks upon surgery). The transplants were fixed in freshly prepared 4% paraformaldehyde in PBS, and demineralized in 10% EDTA, pH 8.0, and embedded in paraffin. Sections were prepared in 5-7 μm thickness and stained with hematoxylin and eosin according to the standard procedures.

### Results

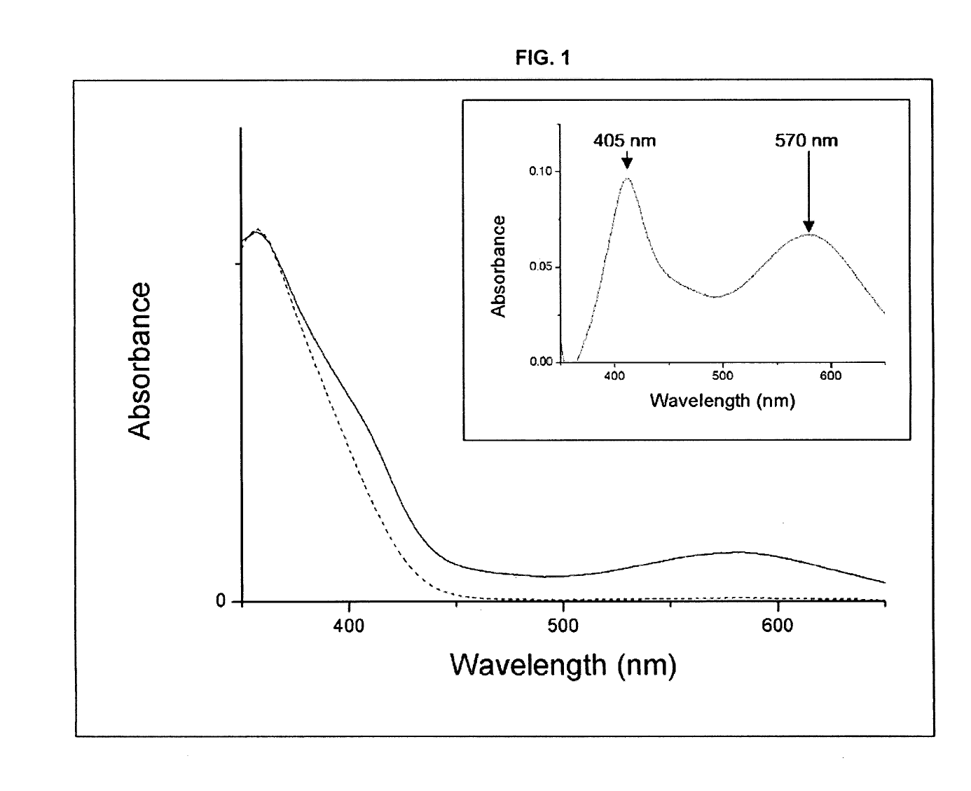

### FIG. 1.

After refluxing HA powders with APS in toluene, HA powders were washed several times with toluene and then with ethanol. Subtraction of spectrum before reaction (FIG. 1. dashed line) from the one after reaction (solid line) indicated increased absorbance at 405 nm and 570 nm (FIG. 1. inset). Increase absorbance at 405 nm and 570 nm indicate Ruheman's purple was produced after ninhydrin reaction. This result shows that APS compound reacted with hydroxyl group on HA and grafted on surface of HA. The surface modification of HA and APS demonstrate that surface of HA can be covalently modified with any tri-methoxy-silane or tri ethoxy-silane group-containing compound so that desired chemical groups can be exposed on surface of HA crystals.

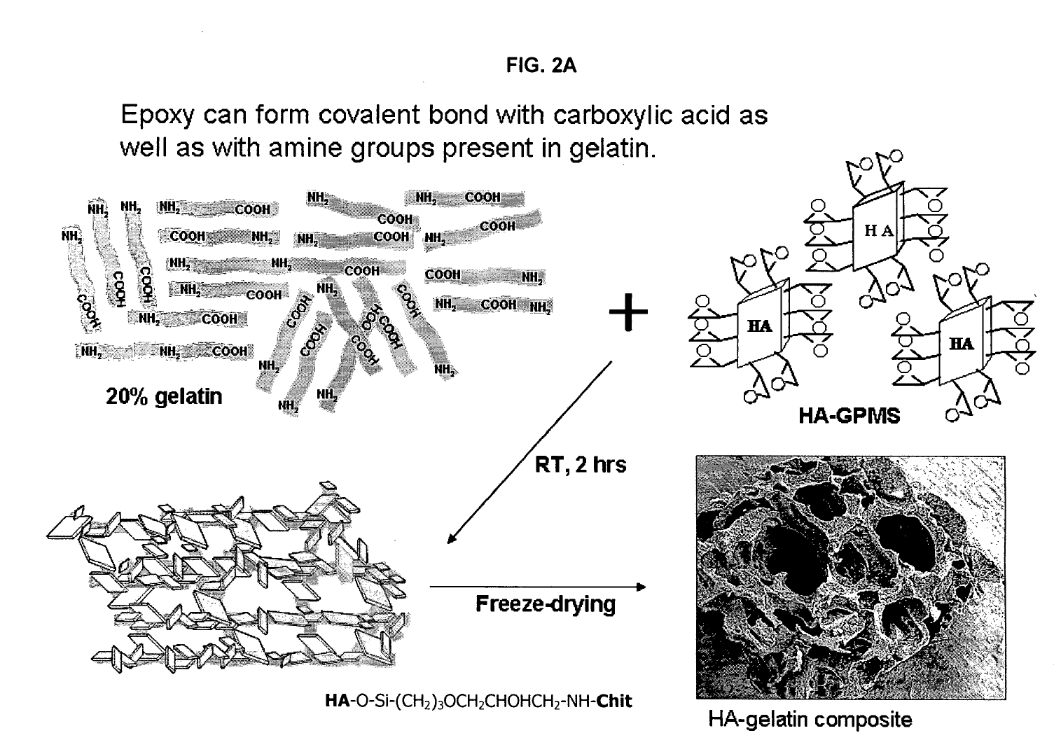

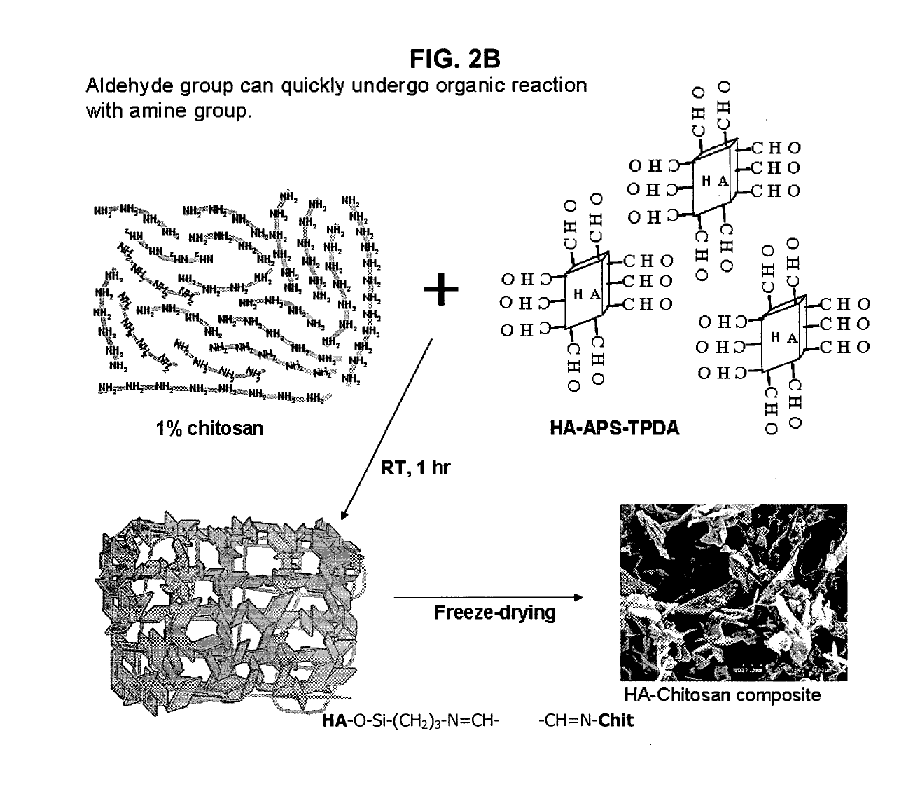

### FIG. 2.

The results of the nihydrin reaction indicate that linker molecules can readily self-assemble on the HA surface through the triethoxysilane group so that the desired functional group can be exposed on surface of HA as indicated in FIG. 2. Presence of amine groups allow subsequent attachment of another linker molecule on the amine-coated HA surface according to the scheme shown in FIG. 2B. FIG. 2B shows two-step process and FIG. 2A shows one-step process of HA-surface modification. The resulting covalent chemical backbone structure is shown below the reaction scheme.

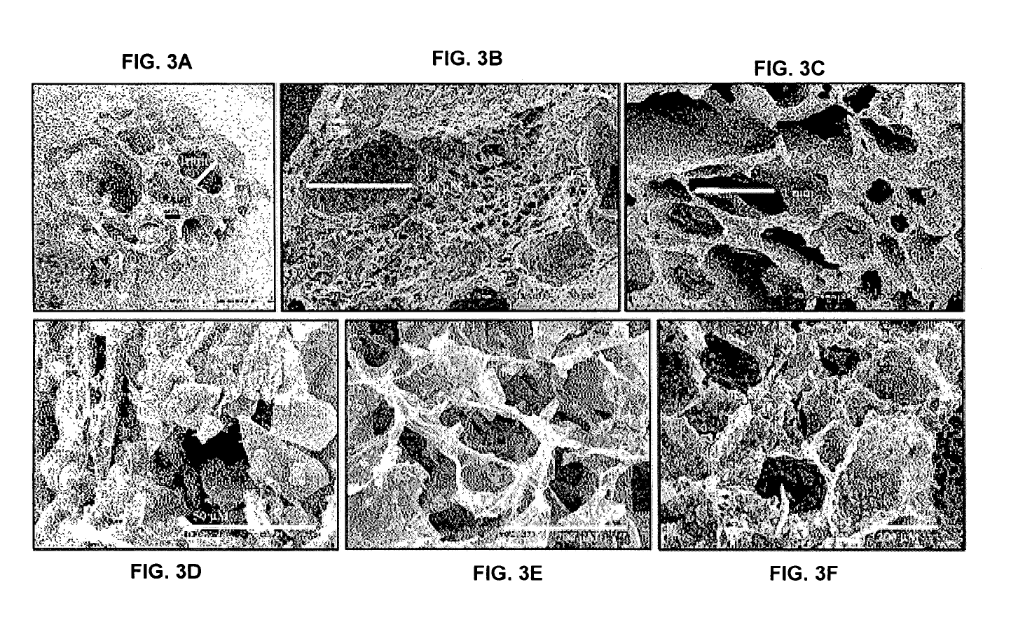

### FIG. 3.

SEM images of composites constructed with HA-GPMS and gelatin (FIGS. 3A, 3B, 3C) and HA-APS-TPDA and chitosan (FIGS. 3D, 3E, 3F). The composite composed of HA-GPMS and gelatin showed biphasic pore size: macroscopic pore ranges 350 μm-1 mm, and microscopic pore ranges around 50 μm. The composites composed of HA-APS-TPDA and chitosan has pores size range ˜200 μm.

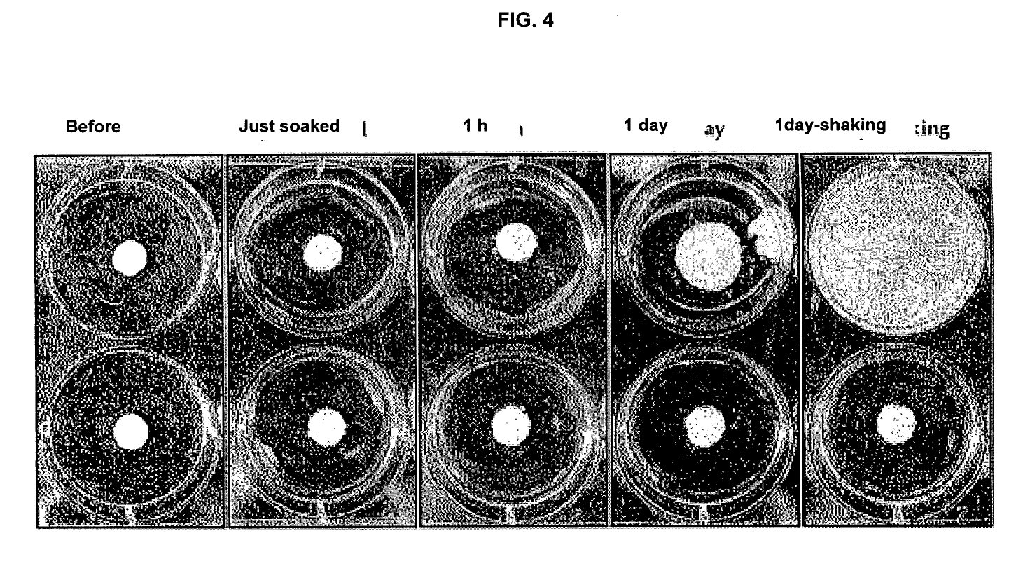

### FIG. 4.

Test of Structural Integrity in Submerged State.

Bare HA-based composites and HA-APS-TPDA-based composites were cut to the same size with 8 mm biopsy punch. Both specimen were submerged in distilled water and checked for their structural integrity over time. After one day, the bare HA-based composite were completely released into the distilled water, while the HA-APS-TPDA composites maintained their structural integrity.

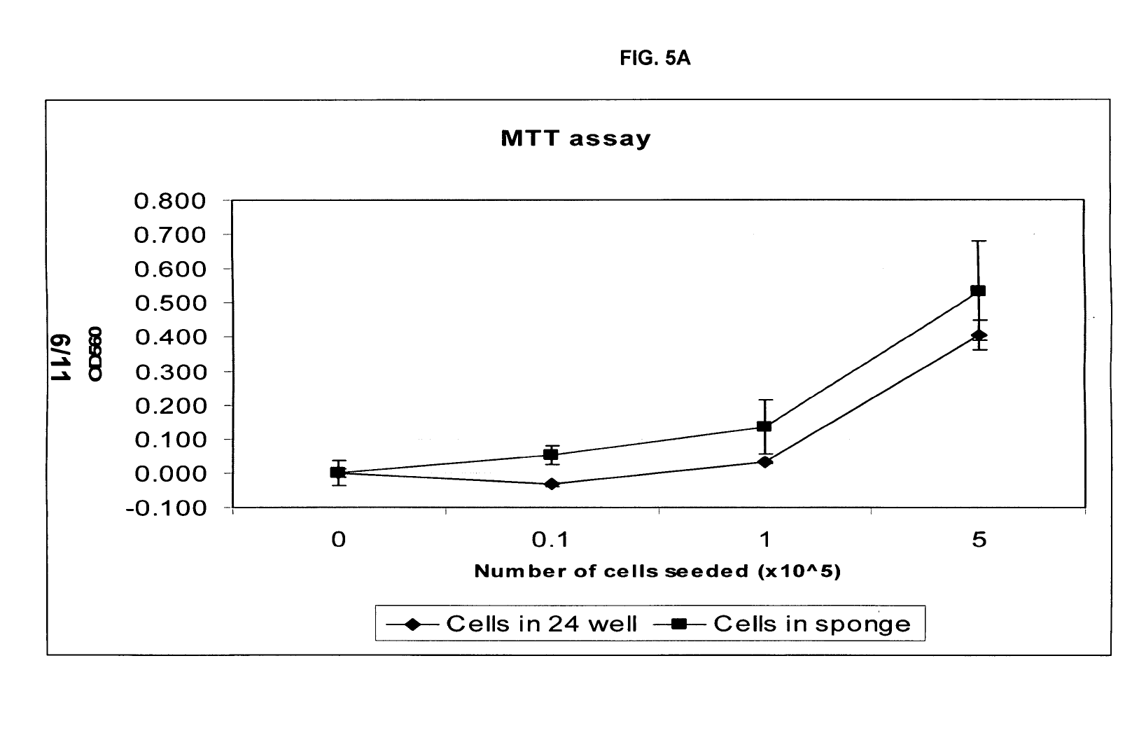

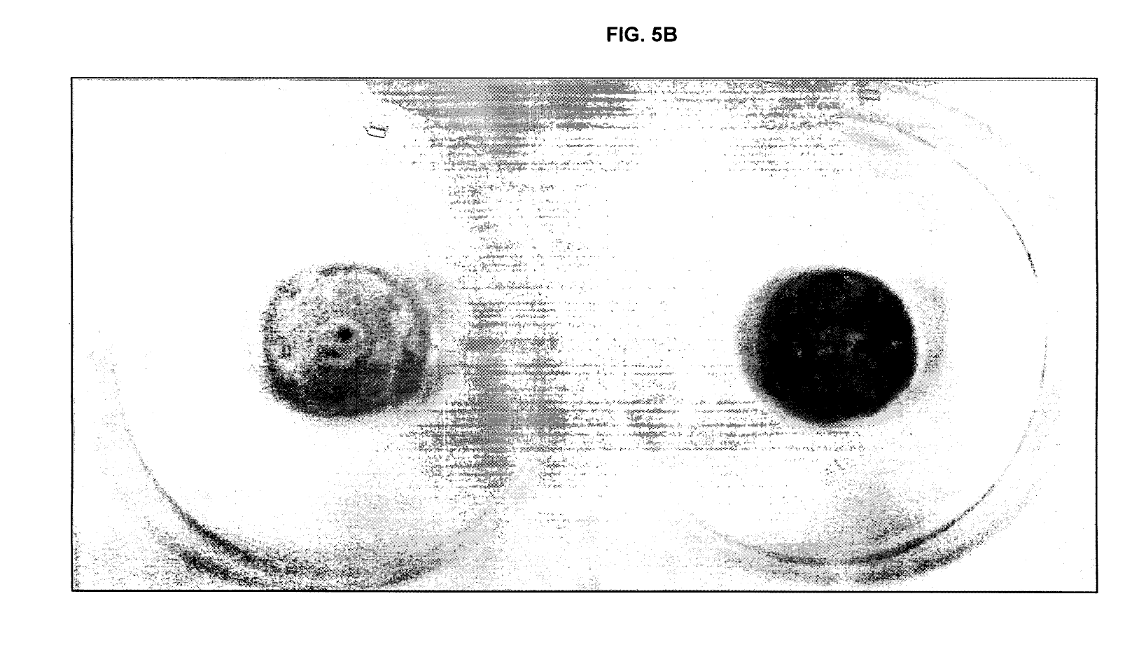

### FIG. 5.

MTT assay was performed to check cytotoxicity of the composite (FIG. 5A). Human bone marrow stromal cells (hBMSCs) were seeded as indicated numbers into the composites and cultured for 24 hours before incubating with MTT dye. The same number of cells were seeded to 24-well without composites and subjected to MTT assay. After incubation with MTT dye, composites were crushed to powder and centrifuged to take supernatant to check optical density. Cells seeded to the composites showed slight increase compared with the cells seeded on 24-well. Cells cultured in the composite increased due to the more available surface area. However, the difference of cell growth was also shown with a low number of cells, as low as 1×10⁴ cells/24 well. Considering that it is not fully confluent density, it is presumed that the materials comprising the composites were not toxic to the cells at all and even better than the plastic surface. Presence of cells in the composite is shown by MTT staining of the composites. Composites with (right well) and without human bone marrow stromal cells (left well) were cultured for 3 weeks and metabolically stained with MTT dye.

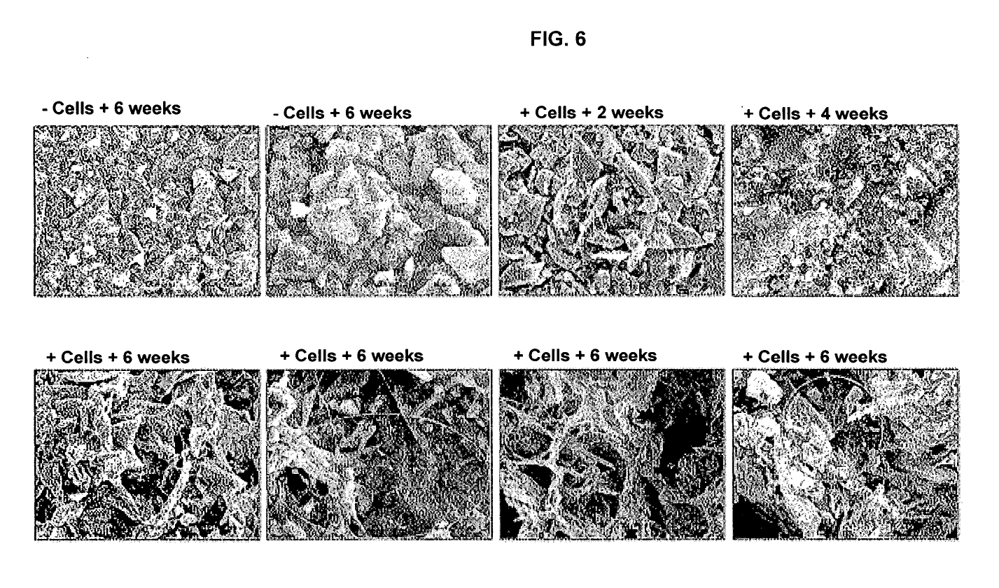

### FIG. 6.

Human bone marrow stromal cells were cultured on composites constructed as 20:1 weight ratio of HA-APS-TPDA:chitosan. The results show that the cells were attached to the composite and formed interconnected network on the composite. At 4 weeks, the seeded hBMSCs started to make granule-like nodules on the surface. This granule-like structure also was shown in other reports and was explained to be Ca-rich granules which contribute to bone matrix formation. At 6 weeks of culture, cell-matrix layer covered the composite surface and formed mesh-like structure between backbones of the composite. This result indicates that the cells seeded on the composite can produce not only ECM proteins but also mineral matrix.

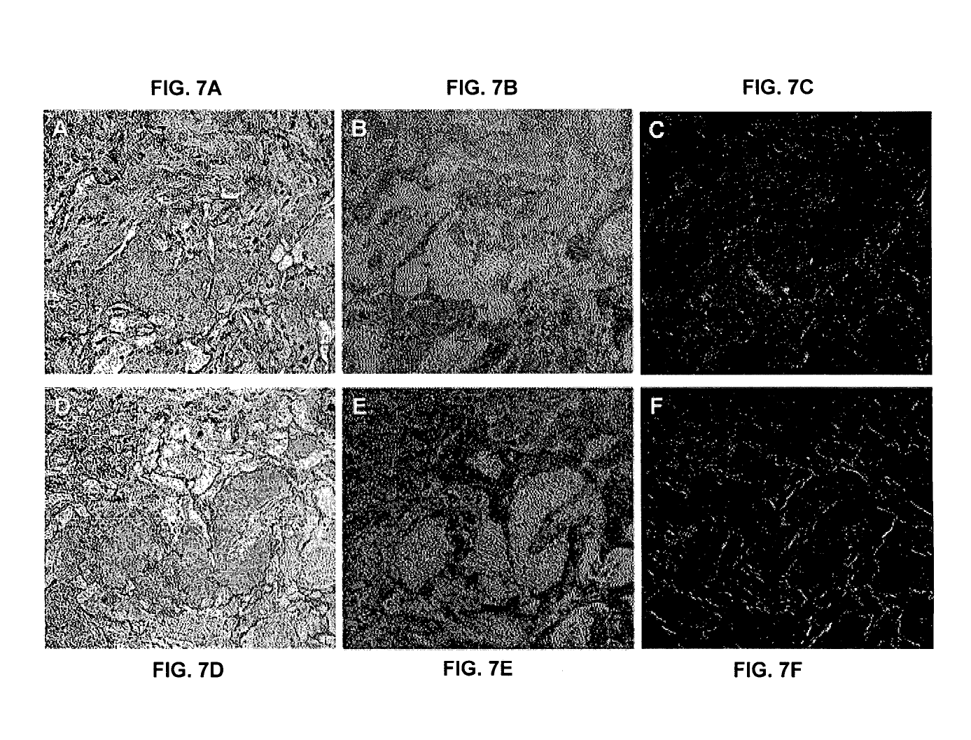

### FIG. 7.

2×10⁶ hBMSCs were seeded to 50 mg of composites constructed as 20:1 weight ratio of HA-APS-TPDA: chitosan, and subcutaneously transplanted to immunocompromised mice. The transplants were collected over time, and examined by histological staining. After 12 weeks, bone-like structure was observed (FIGS. 7A and 7D). When it was observed under UV, auto-fluorescence was shown indicating that the structure was bone (FIGS. 7B and 7E). Based on the fact that the structure polarizes the light under polarized microscope, it is expected that the bone started to form lamellar bone from woven bone at about 12 weeks of in vivo transplantation.

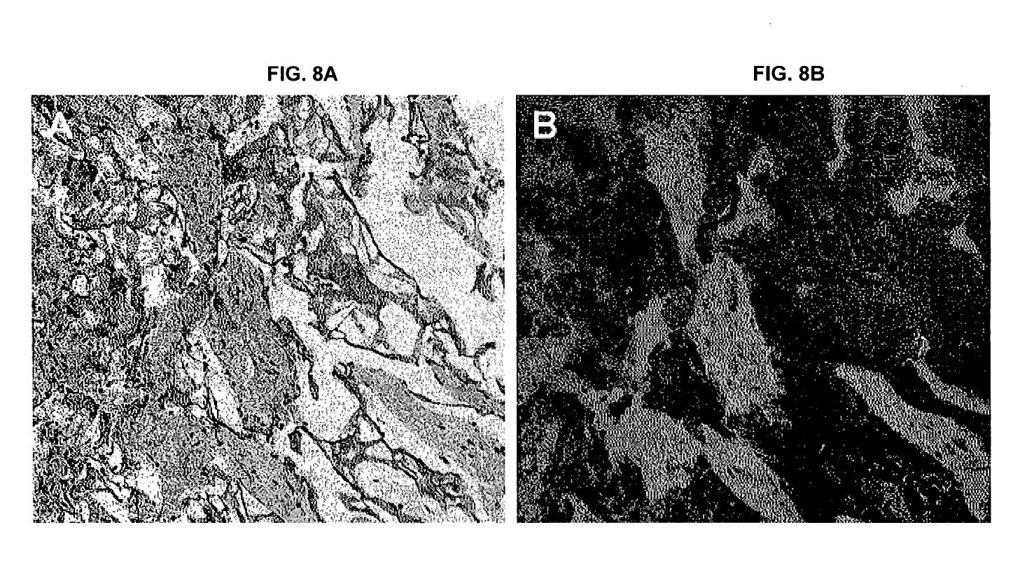

### FIG. 8.

The composites induced formation of bone-like structure after in vivo transplantation even without cells. The composites constructed as 20:1 weight ratio of HA-APS-TPDA: chitosan was subcutaneously transplanted to immunocompromised mice, and the samples were collected over time. Bone-like structure was found in the samples of 12˜15 weeks upon transplant and it autofluoresced under UV light. However, this structure didn't polarize the light under polarized microscope indicating that the bone formation process is delayed in those composites compared to the BMSCs-seeded ones. The host circulating skeletal stem cells might have attached to the composite and helped to induce bone formation. This result shows that the composite has good osteo-inductivity.

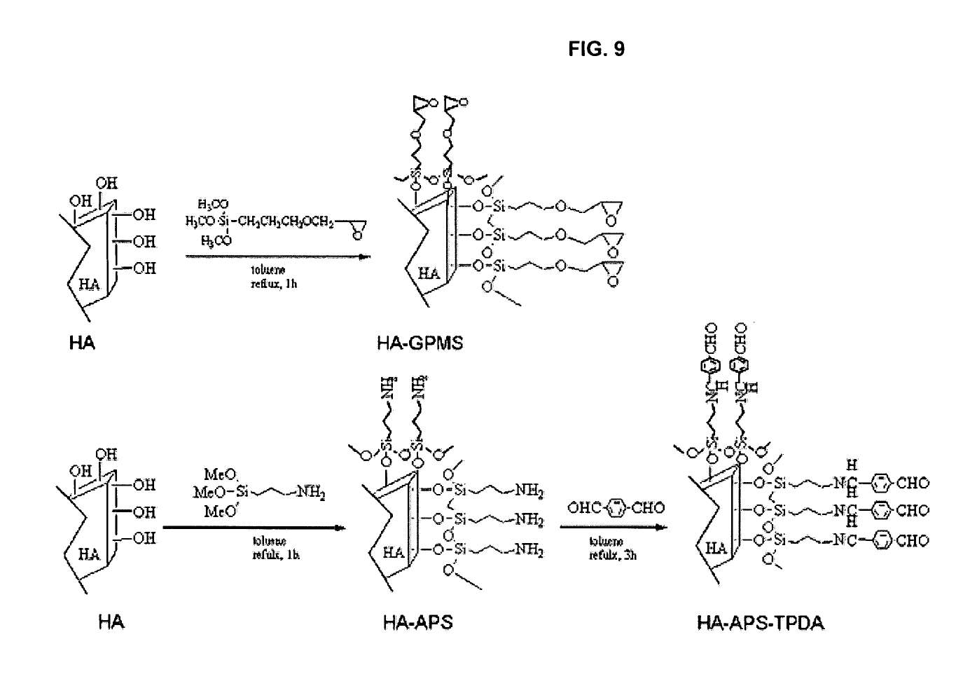

### FIG. 9.

Scheme of the Reaction.

HA can be functionalized with trimetholxysilane or triethoxysilane group-containing molecular linkers. In a one-step process, GPMS was introduced onto the surface of HA resulting in HA covered with epoxy groups (FIG. 9. upper panel). In a two-step process, HA was first functionalized with APS to make HA covered with amine groups which can form covalent linkages with aldehyde. The secondary reaction with dialdehyde compounds such as TPDA resulted in HA covered with aldehyde groups (FIG. 9. lower panel).

These results indicate that the HA-chitosan composites provide an appropriate environment for hBMSCs to survive and undergo biomineralization.

In Vivo Results with HA-Gelatin Composite
4 Weeks after Transplant:
Without cells—small islands of bone-like substance that exhibited autofluorescent in UV light.
Peripheral areas which appear to be possibly bone but not autofluorescent.
With hBMSCs—few areas of bony substance. Not autofluorescent
8 Weeks after Transplant:
Without cells—small bony islands that exhibited autofluorescent˜slightly autofluorescent
With hBMSCs—possibly bony substance, slightly autofluorescent.
10 Weeks after Transplant:
Without cells—the small bone islands had essentially disappeared.
With hBMSCs—no bone.
15 Weeks after Transplant:
Without cells—no visible signs of bone formation.
With hBMSCs—no visible signs of bone formation.

In summary, small bony islands were found in the early stage samples (4-8 weeks), but not in the late stage samples. These initial results indicate that gelatin is not a preferred polyamine polymer, and that chitosan provides comparatively superior results.

In view of the many possible embodiments to which the principles of the disclosed material and methods may be applied, it should be recognized that the illustrated embodiments are only preferred examples and should not be taken as limiting the scope of the invention.

1. A method of bone repair, comprising applying to a subject a composite that comprises:
at least one calcium phosphate;
at least one polyamine polymer material; and
a linking structure covalently crosslinking the calcium phosphate to the polyamine polymer material,
wherein the linking structure is covalently bonded to the calcium phosphate via a —O—Si— moiety or an isocyanato moiety and the linking structure is covalently bonded to the polyamine polymer material via amine groups of the polyamine polymer.

2. The method of claim 1, wherein the calcium phosphate comprises hydroxyapatite and the polyamine polymer comprises chitosan.

3. The method of claim 1, wherein the composite further comprises mesenchymal stem cells.

4. The method of claim 1, wherein the composite further comprises human bone marrow stromal cells.

5. The method of claim 1, wherein the linking structure has a structure represented by:
—O—Si-link-N(H)ₐ—
wherein a is 0 or 1;
“link” is a residual structure derived from a bifunctional linker compound; and
“link” is covalently bonded to the nitrogen atom via a single or double bond.

6. The method of claim 5, wherein the bifunctional linker compound includes:
(i) at least one first functional group selected from a silyl group, an alkoxysilyl group, or an isocyanato group; and
(ii) at least one second functional group selected from carbonyl, carboxyl, or epoxy.

7. The method of claim 5, wherein the bifunctional linker compound is selected from at least one of:
R₃Si-L-X (Formula 1)
R₃Si-L-Y (Formula 2)
Y-L-Y (Formula 3)
Y—Y (Formula 4)
R₃Si-L (Formula 5)
wherein R represents a halogen atom, or a C₁-C₄ alkoxy or a C₁-C₄ alkyl group, provided at least one of the three R groups is a halogen atom or an alkoxy group; L represents substituted or unsubstituted C₁-C₁₇ alkyl, aralkyl or aryl group which may have at least one oxygen, nitrogen and sulfur atom; X represents a leaving group selected from the group consisting of halogen, isocyanate, tosyl and azide; Y represents a reactive functional group of coordinate compounds capable of exchanging ligands selected from the group consisting of hydroxyl, thiol, amine, ammonium, sulfone and its salt, carboxyl acid and its salt, acid anhydride, epoxy, aldehyde, ester, acrylate, isocyanate (—NCO), sugar residue, double bond, triple bond, diene, diyne and alkylphosphine.

8. The method of claim 1, wherein the linking structure is covalently bonded to the calcium phosphate via a —O—Si— moiety.

9. The method of claim 1, wherein the composite is osteoinductive.

10. The method of claim 1, wherein the composite further comprises at least one osteogenic agent.

11. A method of bone repair, comprising introducing into a subject a composite that comprises:
at least one bone replacement material having surface-exposed hydroxyl groups;
at least one polyamine polymer material; and
a linking structure covalently crosslinking the calcium phosphate to the polyamine polymer material,
wherein the linking structure is covalently bonded to the calcium phosphate via a —O—Si— moiety or an isocyanato moiety and the linking structure is covalently bonded to the polyamine polymer material via amine groups of the polyamine polymer.

12. A method of bone repair, comprising injecting into a subject:
at least one polyamine polymer; and
calcium phosphate particles that are substantially coated with at least one functional group that is covalently reactive with amine groups on the polyamine polymer.

13. The method of claim 12, wherein the polyamine polymer and the calcium phosphate particles gel in vivo resulting in a porous three-dimensional composite in which the calcium phosphate particles are substantially immobilized within a crosslinked polyamine polymer matrix.

14. The method of claim 12, wherein the polyamine polymer and the calcium phosphate particles are mixed together prior to injection.

15. The method of claim 12, wherein the polyamine polymer and the calcium particles are mixed together during injection.

16. The method of claim 12, wherein the polyamine polymer and the calcium particles are injected separately.

17. The method of claim 12, wherein the calcium phosphate comprises hydroxyapatite and the polyamine polymer comprises chitosan.

18. The method of claim 12, wherein the functional group coating the calcium phosphate particles is selected from carbonyl, carboxyl, or epoxy.

19. The method of claim 17, wherein the functional group coating the calcium phosphate particles is epoxy.

20. The method of claim 12, wherein the functional group coating the calcium phosphate particles is covalently bonded to the calcium phosphate via a —O—Si— moiety.

21. The method of claim 12, wherein the injection is via a syringe.

22. The method of claim 12, wherein the polyamine polymer is suspended or dissolved in a liquid carrier, and the calcium phosphate particles are suspended to dissolved in a liquid carrier.

23. A composite comprising:
at least one calcium phosphate;
at least one polyamine polymer material; and
a linking structure covalently crosslinking the calcium phosphate to the polyamine polymer material,
wherein the linking structure is covalently bonded to the calcium phosphate via a —O—Si— moiety or an isocyanato moiety and the linking structure is covalently bonded to the polyamine polymer material via amine groups of the polyamine polymer.

24. The composite of claim 23, wherein the calcium phosphate comprises hydroxyapatite, and the polyamine polymer comprises chitosan.

25. The composite of claim 23, wherein the linking structure is covalently bonded to the polyamine polymer material via a —CH₂—NH— moiety or a —CH═N— moiety.

26. The composite of claim 23, wherein the composite further comprises mesenchymal stem cells.

27. The composite of claim 23, wherein the composite further comprises human bone marrow stromal cells.

28. The composite of claim 23, wherein the linking structure has a structure represented by:
—O—Si-link-N(H)ₐ—
wherein a is 0 or 1;
“link” is a residual structure derived from a bifunctional linker compound; and
“link” is covalently bonded to the nitrogen atom via a single or double bond.

29. The composite of claim 28, wherein the bifunctional linker compound includes:
(i) at least one first functional group selected from a silyl group, an alkoxysilyl group, or an isocyanato group; and
(ii) at least one second functional group selected from carbonyl, carboxyl, or epoxy.

30. The composite of claim 28, wherein the bifunctional linker compound is selected from at least one of:
R₃Si-L-X (Formula 1)
R₃Si-L-Y (Formula 2)
Y-L-Y (Formula 3)
Y—Y (Formula 4)
R₃Si-L (Formula 5)
wherein R represents a halogen atom, or a C₁-C₄ alkoxy or a C₁-C₄ alkyl group, provided at least one of the three R groups is a halogen atom or an alkoxy group; L represents substituted or unsubstituted C₁-C₁₇ alkyl, aralkyl or aryl group which may have at least one oxygen, nitrogen and sulfur atom; X represents a leaving group selected from the group consisting of halogen, isocyanate, tosyl and azide; Y represents a reactive functional group of coordinate compounds capable of exchanging ligands selected from the group consisting of hydroxyl, thiol, amine, ammonium, sulfone and its salt, carboxyl acid and its salt, acid anhydride, epoxy, aldehyde, ester, acrylate, isocyanate (—NCO), sugar residue, double bond, triple bond, diene, diyne and alkylphosphine.

31. The composite of any ene of claim 23, wherein the linking structure is covalently bonded to the calcium phosphate via a —O—Si— moiety.

32. The composite of claim 23, wherein the composite is osteoinductive.

33. The composite of claim 23, wherein the composite further comprises at least one osteogenic agent.

34. The composite of claim 23, wherein the composite includes pores ranging in size from about 5 μm to about 1 mm.

35. A bone implant material comprising a composite comprising:
at least one calcium phosphate;
at least one polyamine polymer material; and
a linking structure covalently crosslinking the calcium phosphate to the polyamine polymer material,
wherein the linking structure is covalently bonded to the calcium phosphate via a —O—Si— moiety or an isocyanato moiety and the linking structure is covalently bonded to the polyamine polymer material via amine groups of the polyamine polymer.

36. A combination of ingredients comprising:
at least one polyamine polymer; and
calcium phosphate particles that are substantially coated with at least one functional group that is covalently reactive with amine groups on the polyamine polymer;
wherein the ingredients are adapted for injection into a subject for forming a bone replacement material in vivo.

37. The combination according to claim 36, wherein the calcium phosphate comprises hydroxyapatite, and the polyamine polymer comprises chitosan.

38. The combination according to claim 36, wherein the functional group substantially coating the calcium phosphate particles is at least one group selected from carbonyl, carboxyl or an epoxy.

39. The combination according to claim 36, wherein the combination of ingredients gels in vivo resulting in a porous three-dimensional composite in which the calcium phosphate particles are substantially immobilized within a crosslinked polyamine polymer matrix.

40. The combination according to claim 36, wherein the functional group coating the calcium phosphate particles is covalently bonded to the calcium phosphate via a —O—Si— moiety.
